(19) United States
(12) Patent Application Publication
(10) Pub. No.: US 2018/0147312 A1
(43) Pub. Date: May 31, 2018

(54) Ventilation Duct to Eradicate Indoor Odor and Microbes

(71) Applicant: Douglas Lee Ryerson, Modesto, CA (US)

(72) Inventor: Douglas Lee Ryerson, Modesto, CA (US)

(21) Appl. No.: 15/828,121

(22) Filed: Nov. 30, 2017

Related U.S. Application Data
(60) Provisional application No. 62/428,308, filed on Nov. 30, 2016.

(51) Int. Cl. A61L 9/12; A61L 9/20; F24F 7/06

(52) U.S. Cl. CPC A61L 9/122; A61L 9/20; A61L 2209/21; A61L 2209/16; F24F 7/06

(57) ABSTRACT

A ventilation duct to eradicate indoor odor and microbes includes an air purifying enclosure, a hydroxyl generator, a centrifugal duct fan, an air directing duct. The air purification process is completed through the ventilation duct to eradicate indoor odor as the air purifying enclosure, the hydroxyl generator, the centrifugal duct fan, and the air directing duct are in fluid communication with each other. The hydroxyl generator disinfects and converts a flow of contaminant air into a flow of purified air after the flow of contaminant air is drawn into the air purifying enclosure. The flow of purified air is then respectively discharged from the air purifying enclosure to the centrifugal duct fan and the air directing duct, before exiting the ventilation duct.

The current application claims a priority to the U.S. Provisional Patent application Ser. No. 62/428,308 filed on Nov. 30, 2016.

### FIELD OF THE INVENTION

The present invention is apparatus that eliminates odor and microbes in an indoor air space of buildings such as garbage rooms particularly in single or multi-story apartment buildings and any other indoor rooms of commercial buildings with offensive odors or overgrowth of microbes in a confined area that has limited air movement or air circulation. More specifically, the present invention draws the contaminant indoor air and discharges as a purified air flow thus eliminating eradicating order and microbes.

### BACKGROUND OF THE INVENTION

Building indoor garbage room areas, particularly in Apartment buildings but not excluding commercial business buildings, have unwelcome odors, microbes' growth and larva, flies and other insect species. To remove these odors and species, the source that creates these odors and attract species need to be destroyed. In an outdoor environment, these problems are combated through Hydroxyls. More specifically, Hydroxyls are naturally produced in the atmosphere by the sun each day by photons breaking molecular bonds to convert H20 into H+ and OH— which is a Hydroxyl ion (OH—) or also known as Hydroxide ion.

Hydroxyls are so important that humans could not exist due to Hydroxyls killing efficiency during the day to keep microorganism numbers down which keeps humans' from being infected. Hydroxyls are healthy to breathe since they have been around for millions of years during human evolution. There are on average 2.6 million Hydroxyls per cm³ at noon in the outside air which is when the sun is most effective at producing Hydroxyls from the water vapor in the air. Hydroxyls are very potent and efficient at killing Bacteria, molds and viruses at a higher efficiency level than Ozone or Chlorine. Humans breath Hydroxyls in every breathe outside since the sun is producing them constantly, and the cascade effect on oxygen in the atmosphere creates even more production of Hydroxyls when the sun is shining. However, when the sun is set, Hydroxyl generation process is dropped to zero since Hydroxyls have a half-life that is very short. Even though existing apparatus and systems may claim to be an indoor garbage room odor and microbe destroyer, these existing apparatus and system only provide a temporally solution for the problem and fail to address the source of the problem.

It is therefore an objective of the present invention to eliminate odor and microbes in an indoor air space of building. The present invention produces Hydroxyls in an indoor environment, same as nature does outdoors, to oxidize Volitile Organic Chemicals (VOC) that create the odors. More specifically, The VOCs are broken down into water and carbon dioxide by the Hydroxyls purifying the surrounding air space. Hydroxyls also eradicate microbes, bacteria, molds and viruses. Furthermore, the indoor Hydroxyls are Food and drug administration (FDA) approved and safe for indoor areas where people can work and benefit from a more aseptic environment. Indoors oxygen level rise to on average 21% when the present invention is implemented as the indoor oxygen level ranges normally range from 17-20% upon outside air exchange percentage of Heating, ventilation, and air conditioning (HVAC) systems or opening windows and doors. Since an indoor airspace has zero Hydroxyls and the present invention produces and distributes the Hydroxyls, the present invention is able to mimic the same exact Hydroxyls generation process that completed by the sun. As a result, the present invention can purify 100% of the indoor air volume to provide maximum effect of the Hydroxyls on all surfaces that they come in contact with and also to breakdown VOCs.

### BRIEF DESCRIPTION OF THE DRAWINGS

FIG. 1 is a top perspective view of the present invention.

FIG. 2 is a bottom perspective view of the present invention.

FIG. 3 is a front view of the present invention, showing the plane upon which a cross sectional view is taken shown in FIG. 4.

FIG. 4 is a cross section view of the present invention taken along line 4-4 of FIG. 3.

FIG. 5 is a top perspective view of the air purifying enclosure of the present invention.

FIG. 6 is a bottom perspective view of the air purifying enclosure of the present invention.

FIG. 7 is a front view of the air purifying enclosure of the present invention, showing the plane upon which a cross sectional view is taken shown in FIG. 8.

FIG. 8 is a cross section view of the air purifying enclosure of the present invention taken along line 8-8 of FIG. 7.

FIG. 9 is a perspective view of the centrifugal duct fan of the present invention.

FIG. 10 is a front view of the centrifugal duct fan of the present invention.

FIG. 11 is a basic flowchart illustrating the in-fluid communication of the present invention in relation to the flow of contaminant air and the flow of purified air.

### DETAIL DESCRIPTIONS OF THE INVENTION

All illustrations of the drawings are for the purpose of describing selected versions of the present invention and are not intended to limit the scope of the present invention.

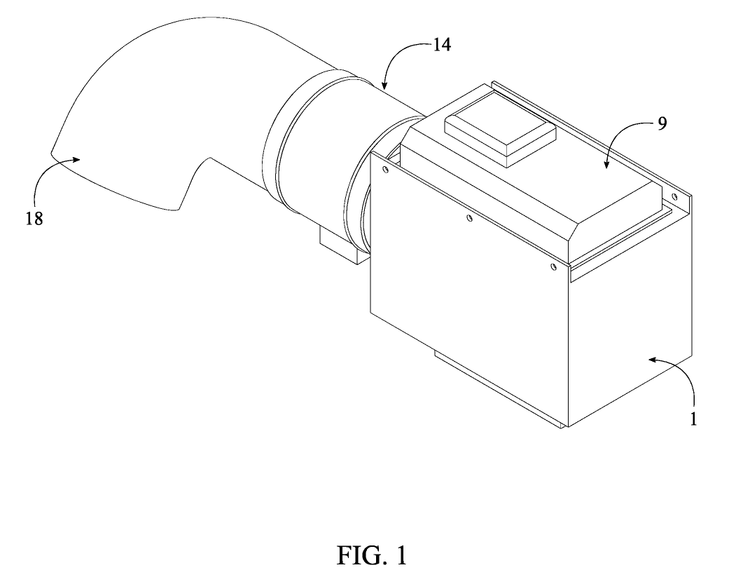

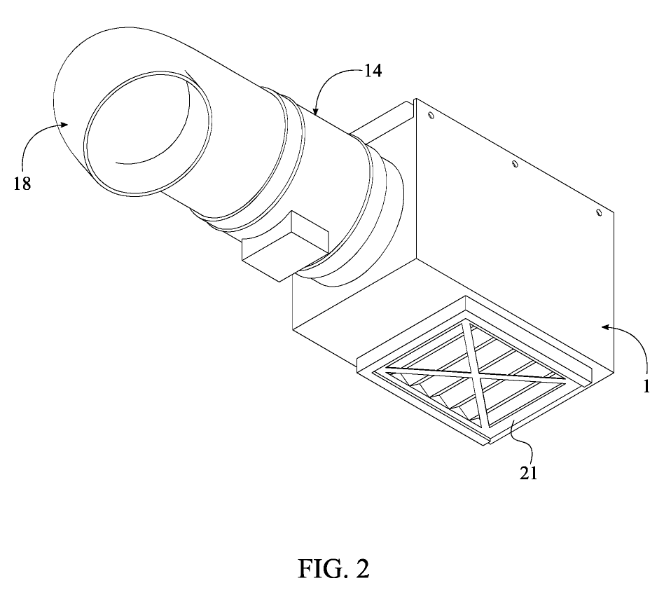

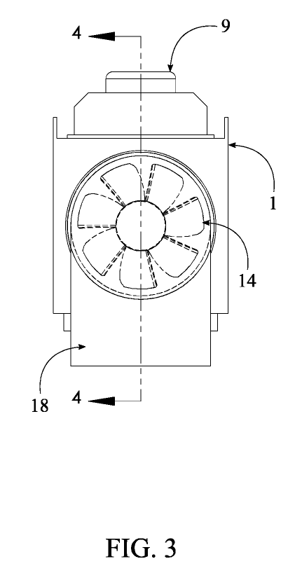

The present invention is a ventilation duct to eradicate indoor odor and microbes within indoor buildings such as garbage areas, basements, and other commercial building indoor spaces. More specifically, the present invention generates and distributes hydroxyl molecules evenly thorough out an indoor environment that can be contaminated with odors, microbes, pests and insects. The hydroxyl molecules can then clean and decontaminate polluted indoor environment by breaking down and neutralizing odor molecules and gases thus repelling microbes, pests, and insects. In order to complete this process, the present invention comprises an air purifying enclosure 1, a hydroxyl generator 9, a centrifugal duct fan 14, and an air directing duct 18 as shown in FIG. 1-3. More specifically, the air purifying enclosure 1, the hydroxyl generator 9, the centrifugal duct fan 14, and the air directing duct 18 are in fluid communication with each other so that a flow of contaminant air can be converted into a flow of purified air through the present invention.

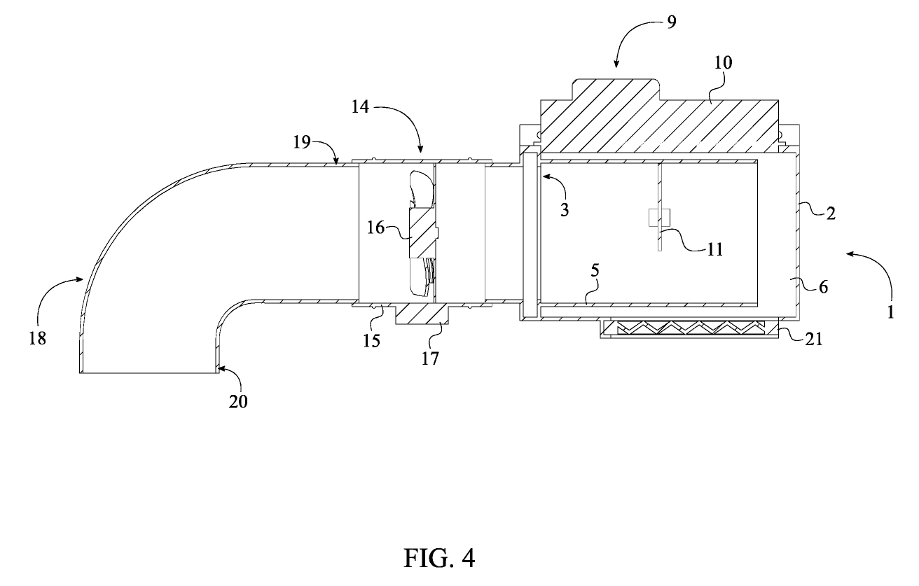

The air purifying enclosure 1 draws the flow of contaminant air into the present invention and discharges the flow of purified air through the operation of the centrifugal duct fan 14. In reference to FIG. 4-6, the air purifying enclosure 1 comprises an enclosure body 2, a separator wall 3, a flow-through chamber 5, an air chamber 6, a contaminant air inlet 7, a purified air outlet 8, and a lateral opening 4. More specifically, the enclosure body 2 functions as the housing of the air purifying enclosure 1 as all of the other components of the air purifying enclosure 1 are based upon the enclosure body 2.

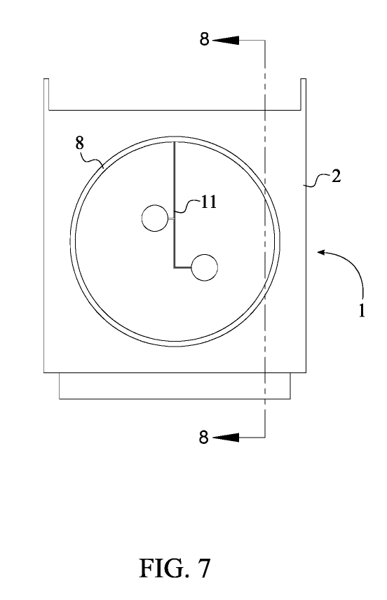

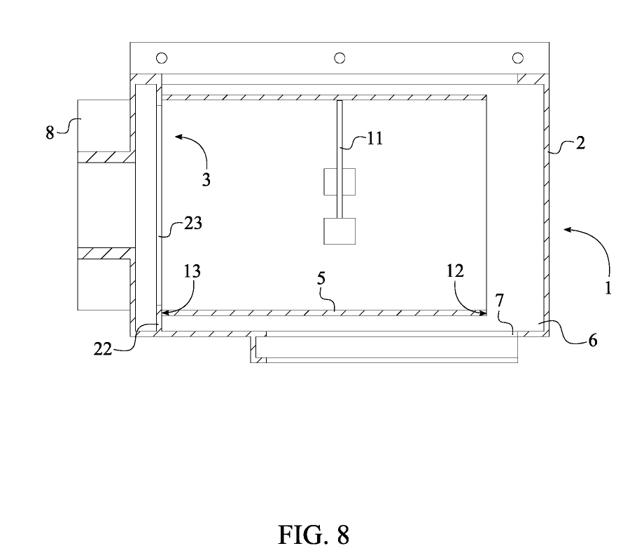

The separator wall 3 is a critical component of the present invention as the separator wall 3 is able to isolate the flow of contaminant air and the flow of purified air within the air purifying enclosure 1. Additionally, the separator wall 3 also functions as the attachment wall for the flow-through chamber 5 within the present invention. In reference to FIG. 7-8, the separator wall 3 comprises a wall body 22 and a wall opening 23 so that the flow-through chamber 5 can be internally connected to the enclosure body 2 by the separator wall 3. More specifically, the wall body 22 is hermetically connected to the enclosure body 2 as the wall opening 23 concentrically traverses through the wall body 22. An air outlet 13 of the flow-through chamber 5 is perimetrically positioned around the wall opening 23 and is hermetically connected to the wall body 22. An air inlet 12 of the flow-through chamber 5 is positioned within the enclosure body 2 and extended away from the separator wall 3. As a result, the enclosure body 2, the separator wall 3, and the flow-through chamber 5 delineates the air chamber 6 within the air purifying enclosure 1 as shown in FIG. 8. In other words, the empty space that is created within the air purifying enclosure 1 by the enclosure body 2, the separator wall 3, and the flow-through chamber 5 is identified as the air chamber 6 within the present invention.

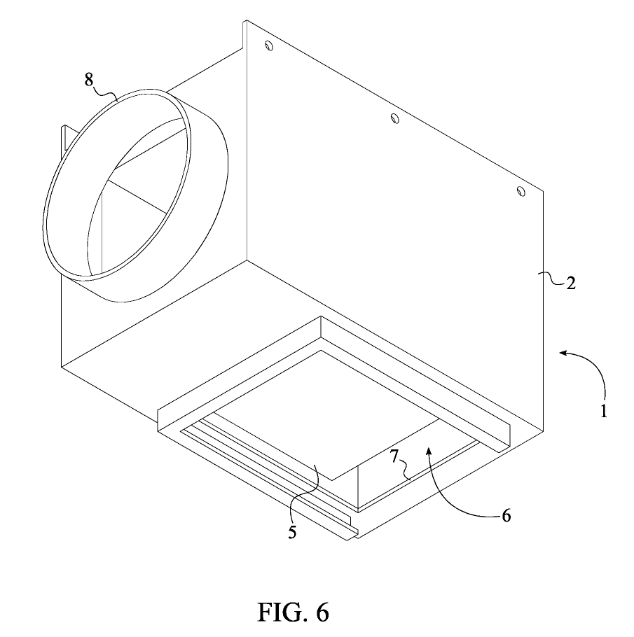

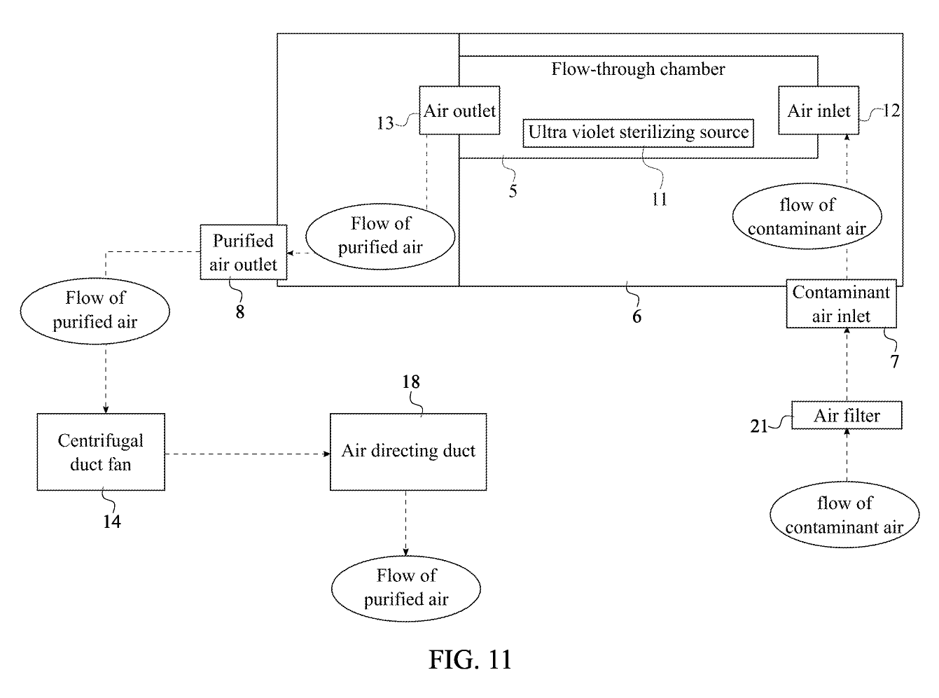

In reference to FIG. 4, FIG. 6, and FIG. 11, the contaminant air inlet 7 traverses into the air chamber 6 through the enclosure body 2. As a result, the contaminant air inlet 7 is in fluid communication with the flow-through chamber 5 through the air chamber 6 so that the flow of contaminant air can be drawn into the air purifying enclosure 1. Furthermore, the contaminant air inlet 7 can be externally covered with an air filter 21 to keep out lager contaminant particle away from the air purifying enclosure 1. More specifically, the air filter 21 is slidably engaged to the enclosure body 2 and positioned adjacent to the contaminant air inlet 7 thus enclosing the contaminant air inlet 7. Since the air filter 21 is sildably engaged with the enclosure body 2, a user or an operator of the present invention can easily change the air filter 21 when necessary.

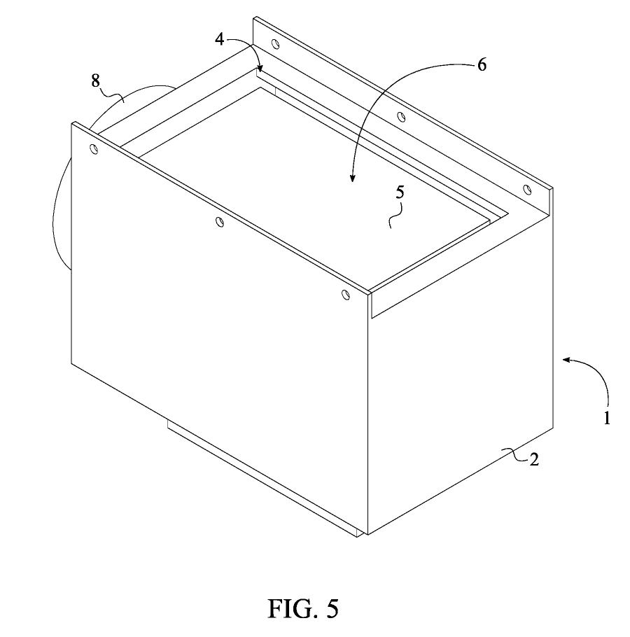

In reference to FIG. 5 and FIG. 11, the purified air outlet 8 traverses into the wall opening 23 through the enclosure body 2. As a result, the purified air outlet 8 is in fluid communication with the flow-through chamber 5 through the separator wall 3 so that the flow of purified air can be discharged from the air purifying enclosure 1.

In reference to FIG. 5, the lateral opening 4 traverses into the air chamber 6 through the enclosure body 2 so that the lateral opening 4 is able to receive the hydroxyl generator 9 with reference to the flow of contaminant air and the flow of purified air. In other words, the hydroxyl generator 9 is in fluid communication with the flow-through chamber 5 within the enclosure body 2 thus enabling the air purification process of the flow of contaminant air into the flow of purified air. The hydroxyl generator 9 first reacts with many Volatile Organic Compounds (VOCs) to remove hydrogen atom thus forming water and an alkyl radical (R*) that typically reacts rapidly with oxygen (O₂) forming peroxy radical (RO₂*). Following is the basic chemical reaction for the aforementioned process;

.OH+RH→H₂O+R.

R.+O₂→RO₂*

In reference to FIG. 4 and FIG. 11, the hydroxyl generator 9 comprises a generator control unit 10 and an ultra violet sterilizing source 11. More specifically, the generator control unit 10 is mounted onto the lateral opening 4 thus isolating the contaminant air inlet 7 as the only opening that draws the flow of contaminant air. The ultra violet sterilizing source 11 is positioned and mounted within the flow-through chamber 5 as the generator control unit 10 is electrically connected to the ultra violet sterilizing source 11. The ultra violet sterilizing source 11 utilizes a disinfection method that uses short-wavelength ultraviolet light to kill or inactivate microorganisms by destroying nucleic acid and disrupting their deoxyribonucleic acid molecules. Since the flow of contaminant air is drawn into the flow-through chamber 5 through the air inlet 12 and the flow of purified air is discharged out of the flow-through chamber 5 through air outlet 13, the air outlet 13 is in fluid communication with the air inlet 12 through the ultra violet sterilizing source 11. The hydroxyl generator 9 is electrically connected to an external power source through the generator control unit 10 so that the ultra violet sterilizing source 11 can be powered within the present invention.

The centrifugal duct fan 14 functions as an air booster unit and the air directing duct 18 functions as a directional outlet for the flow of purified air within the present invention. In reference to FIG. 4 and FIG. 11, the centrifugal duct fan 14 and the air directing duct 18 are positioned external to the enclosure body 2 in such a way that the air directing duct 18 is in fluid communication with the purified air outlet 8 through the centrifugal duct fan 14.

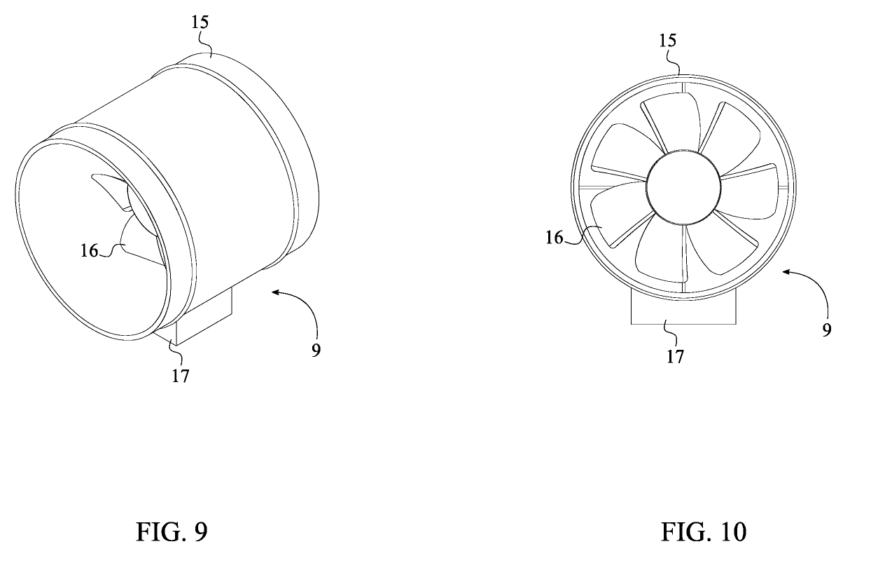

In reference to FIG. 9-10, the centrifugal duct fan 14 comprises an air duct 15, a fan 16, and a fan control unit 17. The fan 16 is concentrically positioned within the air duct 15 and adjacently mounted to the air duct 15. As a result, the air duct 15 functions as a protective sleeve for the fan 16 and an air channel for the flow of purified air. The fan control unit 17 is externally mounted to the air duct 15 and electrically connected to the fan 16 through the air duct 15. The fan control unit 17 draws electricity from and external power source and then electrically powers the fan 16 within the present invention. As a result, the centrifugal duct fan 14 is able to control and maintain the air flow with respect to the flow of contaminant air and the flow of purified air.

In reference to FIG. 4, the air duct 15 is concentrically positioned in between the purified air outlet 8 and an inlet end 19 of the air directing duct 18. More specifically, the inlet end 19 is terminally positioned to the air duct 15 and oppositely positioned of the purified air outlet 8. The inlet end 19 is concentrically and rotatably mounted to the air duct 15 thus maintaining a stationary position for the air duct 15 within the present invention. Resultantly, an outlet end 20 of the air directing duct 18 is positioned opposite of the air duct 15. The air directing duct 18 is preferably formed into an elongated elbow shape duct so that the flow of purified air that is discharged from the air purifying enclosure 1 can be directionally discharged from the outlet end 20. More specifically, a user or an operator of the present invention can selectively rotate the air directing duct 18 in 360-degrees around a central axis of the centrifugal duct fan 14 thus enabling the directional discharge of the flow of purified air.

Furthermore, the present invention comprises a pair of mounting rails that is extended upward from the enclosure body 2. More specifically, the pair of mounting rails is positioned along the lateral opening 4 and positioned opposite of each other about the enclosure body 2. As a result, the pair of mounting rails enables the present invention to be secured to a wall from either side thus allowing a left-side and right-side placement of the air directing duct.

Since the present invention is able to eradicate indoor odor and microbes, the present invention can be utilized within agricultural industry, medical industry, medical marijuana production facilities, and any other industries that require or utilize hydroxyls to eradicate odors. Additionally, the present invention does not produce or emit any pollutants back into the environment thus completing eco-friendly air purification process.

Although the invention has been explained in relation to its preferred embodiment, it is to be understood that many other possible modifications and variations can be made without departing from the spirit and scope of the invention as hereinafter claimed.

What is claimed is:

1. A ventilation duct to eradicate indoor odor and microbes comprises:
an air purifying enclosure;
a hydroxyl generator;
a centrifugal duct fan;
an air directing duct;
the air purifying enclosure comprises an enclosure body, a separator wall, a flow-through chamber, an air chamber, a contaminant air inlet, and a purified air outlet;
the flow-through chamber being internally connected to the enclosure body by the separator wall;
the contaminant air inlet being in fluid communication with the flow-through chamber through the air chamber;
the purified air outlet being in fluid communication with the flow-through chamber through the separator wall;
the hydroxyl generator being in fluid communication with the flow-through chamber within the enclosure body;
the centrifugal duct fan and the air directing duct being positioned external to the enclosure body; and
the air directing duct being in fluid communication with the purified air outlet through the centrifugal duct fan.

2. The ventilation duct to eradicate indoor odor and microbes as claimed in claim 1 comprises:
the separator wall comprises a wall body and a wall opening;
the wall body being hermetically connected to the enclosure body;
the wall opening concentrically traverses through the wall body;
an air outlet of the flow-through chamber being perimetrically positioned around the wall opening;
the air outlet being hermetically connected to the wall body; and
an air inlet of the flow-through chamber being positioned within the enclosure body.

3. The ventilation duct to eradicate indoor odor and microbes as claimed in claim 1, wherein the enclosure body, the separator wall, and the flow-through chamber delineates the air chamber within the air purifying body.

4. The ventilation duct to eradicate indoor odor and microbes as claimed in claim 1 comprises:
the air purifying enclosure further comprises a lateral opening; and
the lateral opening traverses into the air chamber through the enclosure body.

5. The ventilation duct to eradicate indoor odor and microbes as claimed in claim 1, wherein the contaminant air inlet traverses into the air chamber through the enclosure body.

6. The ventilation duct to eradicate indoor odor and microbes as claimed in claim 1 comprises:
an air filter;
the air filter being slidably engaged to the enclosure body, adjacent to the contaminant air inlet; and
the contaminant air inlet being enclosed by the air filter.

7. The ventilation duct to eradicate indoor odor and microbes as claimed in claim 1, wherein the purified air outlet traverses into a wall opening of the separator wall through the enclosure body.

8. The ventilation duct to eradicate indoor odor and microbes as claimed in claim 1 comprises:
the hydroxyl generator comprises a generator control unit and an ultra violet sterilizing source;
the generator control unit being mounted onto a lateral opening of the air purifying enclosure;
the ultra violet sterilizing source being positioned within the flow-through chamber;
the ultra violet sterilizing source being mounted to the flow-through chamber;
the ultra violet sterilizing source being electrically connected to the generator control unit; and
an air inlet of the flow-through chamber being in fluid communication with an air inlet of the flow-through chamber through the ultra violet sterilizing source.

9. The ventilation duct to eradicate indoor odor and microbes as claimed in claim 1 comprises:
the centrifugal duct fan comprises an air duct, a fan, a fan control unit;
the air duct being concentrically mounted to the purified air outlet;
the fan being concentrically positioned within the air duct;
the fan being adjacently mounted to the air duct;
the fan control unit being externally mounted to the air duct; and
the fan being electrically connected to the fan control unit through the air duct.

10. The ventilation duct to eradicate indoor odor and microbes as claimed in claim 1 comprises:
an inlet end of the air directing duct being terminally positioned to the air duct of the centrifugal duct fan, opposite of the purified air outlet;
the inlet end being concentrically and rotatably mounted to the air duct; and
an outlet end of the air directing duct being oppositely positioned to the air duct.
